(19) United States
(12) Patent Application Publication
(10) Pub. No.: US 2020/0000721 A1
(43) Pub. Date: Jan. 2, 2020

(54) AQUEOUS COMPOSITIONS AND ASSOCIATED CREAM FORMULATIONS

(71) Applicant: MedReleaf Corp., Toronto (CA)

(72) Inventor: Alexia Blake, Markham (CA)

(21) Appl. No.: 16/023,628

(22) Filed: Jun. 29, 2018

(51) Int. Cl. A61K 9/06; A61K 47/32; A61K 47/10; A61K 47/14; A61K 47/26; A61K 36/185; A61K 31/05

(52) U.S. Cl. CPC A61K 9/06; A61K 47/32; A61K 47/10; A61K 31/05; A61K 47/26; A61K 36/185; A61K 47/14

(57) ABSTRACT

The disclosure provides aqueous compositions comprising a rheology modifier comprising a water-swelling polymer and a co-emulsifier that are suitable for preparing cream formulations containing an oil. In one example, the composition comprises PPG-3 benzyl ether ethylhexanoate, glycerin and a rheology modifier comprising sodium polyacrylate, ethylhexyl cocoate, PPG-3 benzyl ether myristate, a co-emulsifier such as polysorbate 20, and an antimicrobial agent. Cream formulations can be prepared by mixing the aqueous composition and an oil without heating and provide desirable characteristics for topical use such as oil capacity and skin feel. The disclosure also provides associated methods as well as uses of the compositions for treating a medical condition.

### FIELD

The disclosure relates to aqueous compositions and more specifically aqueous compositions suitable for preparing cream formulations containing an oil for topical use.

### BACKGROUND

Cannabidiol (CBD) is one of several cannabinoids found in cannabis. Cannabidiol has been shown to have a variety of health benefits, including but not limited to reducing anxiety and depression, relieving pain from arthritis, and treating psoriasis. Possible dosage forms for cannabidiol include oils, ointments, capsules, sprays as well as dried cannabis flower for smoking and vaping. Cannabis plant extracts containing cannabinoids such as CBD and/or THC are typically diluted in a carrier oil such as olive oil or sunflower oil. For topical applications, creams are often preferred over oils and ointments, as creams are not as sticky and are easier to apply.

Oil containing creams are typically prepared by forming an emulsion with an aqueous base composition. However, forming a stable emulsion can be difficult due to the immiscible nature of oil and water and the tendency for emulsions to breakdown over time leading to separation. Furthermore, formulations containing oils such as plant extracts can be difficult to produce or require specialized equipment, preventing users from mixing or incorporating oils into creams at home or outside of a manufacturing facility. In addition, emulsions containing high levels of oil often have poor skin feel, limiting their use or desirability as topical creams.

For example, many emulsions formed using classical amphiphilic emulsifiers like Tween® (polysorbates) or Span® (sorbitans) require heating oil and water phases to temperatures near 70-75° C., followed by recombination and mixing under shear. Elevated temperatures are required to solubilize the emulsifiers so that they are mobile and can self-assemble around oil droplets. However, upon cooling, they transition into a solid layer (mono, bilayer) around oil droplets. The resulting emulsion network is rigid, meaning that oil cannot be added to it after it has cooled. Furthermore, once the emulsion breaks down, a process which is accelerated by high shear or high temperatures, the emulsion cannot be reconstituted via mixing. For many users, the ability to prepare cream formulations containing high levels of oil using a ready-made base would be a significant advantage.

While the use of rheology modifiers containing sodium polyacrylate has been described for emulsifying oils, even subtle modifications or the addition of other components may cause formulations to loose desirable properties such as stability and/or skin feel. Maintaining a desirable skin feel can be especially problematic for cream formulations containing high levels of oil.

Accordingly, there is a need for novel compositions and associated formulations containing an oil that have a desirable skin feel and can be prepared easily without heating.

### SUMMARY

In one aspect, there is provided an aqueous composition suitable for preparing an oil-containing cream formulation. The products described herein are capable of containing high levels of oil and can be prepared manually by combining a pre-made aqueous composition and oil without heating and/or extended mixing. Furthermore, the oil containing cream formulations can easily be reconstituted without heating or extended mixing if any separation of oil and the aqueous phase occurs. The products described herein also have desirable sensory characteristics such as skin feel and are easily applied making them particularly suitable for subjects wishing to prepare oil-containing cream formulations for topical use. For example, in one embodiment the aqueous composition is useful for preparing cream formulations for personal use that contain plant extracts with medicinal properties such as cannabidiol (CBD) and/or tetrahydrocannabinol (THC).

As demonstrated in the Examples, aqueous compositions comprising a rheology modifier comprising sodium polyacrylate and a co-emulsifier such as polysorbate 20, as well as glyercin and PPG-3 Benzyl ether ethylhexanoate were found to produce cream formulations with both a high oil load capacity and desirable sensory characteristics such as skin feel. Furthermore, the cream formulations described herein can readily be prepared or reconstituted without heating or extended mixing. Subjects provided the aqueous composition and instructions for preparing cream formulations with CBD oil reported that the cream formulations were “very effective” or “somewhat effective” for treatment of a variety of medical conditions including but not limited to arthritis, PTSD, fibromyalgia, eczema, psoriasis and acne.

Accordingly, in one embodiment there is provided an aqueous composition comprising a rheology modifier comprising sodium acrylate and a co-emulsifier, PPG-3 benzyl ether ethylhexanoate, glycerin and water. Optionally, the aqueous composition further comprises an anti-microbial agent.

In one embodiment, the co-emulsifier is a water-soluble emulsifier such as a water soluble non-ionic surfactant. In one embodiment, the non-ionic surfactant is a polysorbate such as polysorbate 20. In one embodiment, the rheology modifier further comprises a carrier oil such as ethylhexyl cocoate and an emollient such as PPG-3 benzyl ether myristate.

In one embodiment, the aqueous composition comprises less than about 0.25% (w/w) of the co-emulsifier, optionally between about 0.01% (w/w) and 0.25% of the co-emulsifier. In another embodiment, the aqueous composition comprises less than about 0.2% (w/w) of the co-emulsifier, optionally between about 0.02% and 0.2% (w/w) of the co-emulsifier. In one embodiment, the co-emulsifier is a non-ionic surfactant.

In one embodiment, the aqueous composition comprises less than about 3.5%, 3%, 2.5%, or 2% (w/w) sodium polyacrylate. In one embodiment, the aqueous composition comprises between about 0.5% and 3.5% (w/w) or between 1% and 2.8% (w/w) sodium polyacrylate, or between 0.8% and 2.5% (w/w) sodium polyacrylate.

In one embodiment, the aqueous composition comprises between 0.01% and 1% (w/w) of an anti-microbial agent. In one embodiment, the aqueous composition comprises less than about 0.1% (w/w) of an anti-microbial agent, optionally between about 0.05 and 0.2% (w/w). In one embodiment, the anti-microbial agent is potassium sorbate.

In one embodiment, the aqueous composition comprises PPG-3 benzyl ether ethylhexanoate. In one embodiment, the aqueous composition comprises less than about 5%, 4%, 3% or 2% (w/w) of PPG-3 benzyl ether ethylhexanoate. In one embodiment, the aqueous composition comprises between about 1% and 5% (w/w) or between about 2% and 4% (w/w) PPG-3 benzyl ether ethylhexanoate.

In one embodiment, the aqueous composition comprises glycerin. In one embodiment, the aqueous composition comprises less than about 5%, 4%, 3% or 2% glycerin. In one embodiment, the aqueous composition comprises between about 1% and 5% (w/w) or between about 2% and 4% (w/w) glycerin.

In one embodiment, the aqueous composition comprises a rheology modifier comprising sodium acrylate and a co-emulsifier, optionally a non-ionic surfactant. In some embodiments, the rheology modifier may also include an oil such as ethylhexyl cocoate and/or emollient such as PPG-3 benzyl ether myristate. In one embodiment, the rheology modifier comprises, consists essentially of, or consists of: between 50% and 100% (w/w) sodium polyacrylate; between 25% and 50% (w/w) ethylhexyl cocoate; between 5% and 10% (w/w) PPG-3 benzyl ether myristate; and between 1% and 5% (w/w) of a co-emulsifier. In one embodiment, the rheology modifier comprises, consists essentially of, or consists of: between 50% and 69% (w/w) sodium polyacrylate; between 25% and 50% (w/w) ethylhexyl cocoate; between 5% and 10% (w/w) PPG-3 benzyl ether myristate; and between 1% and 5% (w/w) of a co-emulsifier. In one embodiment, the co-emulsifier is a non-ionic water soluble surfactant such as polysorbate 20. In one embodiment, a rheology modifier suitable for use in the aqueous composition described herein is ViscOptima™ SE, commercially available from Croda Inc. (Snaith, UK). In one embodiment, the aqueous composition comprises between 2% and 4% (w/w) of the rheology modifier.

In one embodiment, the aqueous composition comprises, consists essentially of, or consists of: sodium polyacrylate, ethylhexyl cocoate, PPG-3 benzyl ether myristate, polysorbate 20, PPG-3 benzyl ether ethylhexanoate, glycerin, water, and, optionally, an anti-microbial agent. In one embodiment, the aqueous composition comprises, consists essentially of, or consists of: between 2% and 4% (w/w) ViscOptima™ SE, between 2% and 4% w/w PPG-3 benzyl ether ethylhexanoate; between 2% and 4% w/w glycerin; between 0.01% and 1% w/w of an anti-microbial agent; and between 87% and 93.99% (w/w) water.

Also provided are cream formulations comprising an oil and an aqueous composition as described herein. In one embodiment, the oil comprises a plant extract such as cannabidiol (CBD) oil. In one embodiment, the oil comprises a medicinal ingredient, optionally one or more cannabinoids and/or terpenes. In one embodiment, the oil comprises a medicinal ingredient and a carrier oil such as sunflower oil.

In one embodiment, the cream formulation comprises, consists essentially of, or consists of between about 80% and 99% (w/w) of the aqueous composition and between about 1% and 20% (w/w) of the oil. In one embodiment, the cream formulation comprises, consists essentially of, or consists of between about 82% and 95% w/w of the aqueous composition about 5% and 18% (w/w) of the oil. In one embodiment, the cream formulation comprises between 1% and 18% (w/w) oil, optionally between 8% and 18%, between 10% and 18%, or between 12% and 18% (w/w) oil.

Also provided is a method of preparing a cream formulation comprising providing an aqueous composition as described herein and combining the aqueous composition and an oil. In one embodiment, the method comprises manually mixing the aqueous composition and the oil, optionally with a stirring implement such as a rod, spoon, spatula or the like. In one embodiment, the method comprises combining the aqueous composition and the oil without heating to form the cream formulation. In one embodiment, a subject performs the step of combining the aqueous composition and the oil prior to self-administration or use of the cream formulation by the subject.

In one embodiment, there is provided is a method of treating a subject with a medical condition, comprising administering to the subject an effective amount of a cream formulation as described herein. Also provided is the use of a cream formulation as described herein for treating a subject with a medical condition. In one embodiment, the cream formulation is prepared by and/or self-administered by the subject with the medical condition. In one embodiment, the method comprises topically administering to a subject a therapeutically effective amount of a cream formulation comprising CBD oil.

In one embodiment, there is also provided a kit comprising an aqueous composition as described herein and one or more oils. In one embodiment, the oil comprises one or more medicinal ingredients, such as one or more cannabinoids and/or terpenes. In one embodiment, the kit comprises separate containers for the aqueous composition and the one or more oils. In one embodiment, the kit comprises a stirring implement. Optionally, the kit comprises instructions for use, such as for preparing a formulation as described herein and/or for treating a medical condition.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from the detailed description.

### DETAILED DESCRIPTION

The present description provides aqueous compositions suitable for the preparation of oil-containing cream formulations. Also provided are oil-containing cream formulations as well as associated kits and methods. In one embodiment, the cream formulations can be prepared by adding oil to a pre-made aqueous composition without the need for heating or extended mixing. The compositions are also capable of containing high levels of oil while maintaining desirable sensory characteristics for topical use. The aqueous compositions described herein are therefore particularly advantageous for preparing cream formulations for personal use and/or at home using oils with medicinal properties such as cannabidiol (CBD) oil.

Many emulsions or cream formulations rely on the presence of an emulsifying agent; however the preparation of emulsions typically requires specialized conditions such as heating and/or extended mixing to incorporate the oil and form a homogeneous emulsion. Furthermore, emulsion-based formulations often create a rigid network that cannot readily accept oil after cooling, and are therefore difficult to modify or reconstitute once formed or to prepare outside of a manufacturing facility. In contrast, the products described herein contain a rheology modifier comprising sodium polyacrylate and a co-emulsifier that form a fluid network. Oil can be added to the pre-made fluid network to produce a homogenous formulation suitable without heating and/or extended mixing.

In addition, many emulsions produce an undesirable skin feel and/or are difficult to apply, especially for formulations containing high-levels of oil. Oil-containing formulations for topical use should preferably be rapidly absorbed to avoid contact contamination and/or enhance efficacy. As set out in the Examples, products containing glycerin and PPG-3 benzyl ether ethylhexanoate in addition to a rheology modifier comprising sodium polyacrylate and relatively small amounts of a co-surfactant result in cream formulations with a desirable skin feel and rapid absorption of oil without leaving an oily residue on the skin. Notably, the products described herein concurrently are capable of containing high levels of oil, do not require complex emulsification procedures and provide a desirable skin feel.

Accordingly, in one aspect there is provided an aqueous composition comprising glycerin, PPG-3 benzyl ether ethylhexanoate, water, and a rheological modifier comprising sodium polyacrylate and a co-surfactant, optionally a non-ionic surfactant. Optionally, the aqueous composition include one or more of an anti-microbial agent, scent agent and coloring agent.

In one embodiment, sodium polyacrylate in the aqueous composition forms a hydrogel. For example, in one embodiment, the aqueous composition comprises sodium polyacrylate and has a viscosity of between about 40,000 cps and 80,000 cps at 25° C. In one embodiment, the aqueous composition has a viscosity of between about 50,000 cps and 75,000 cps at 25° C., between about 60,000 and 70,000 cps at 25° C., or between about 60,000 and 70,000 cps at 25° C.

The aqueous compositions described herein are structured by the presence of a rheology modifier comprising sodium polyacrylate, which also serves to contain the oil within a homogenous formulation.

In one embodiment, the aqueous composition comprises less than about 4%, 3.5%, 3%, 2.5%, 2%, 1.5%, 1.0%, 0.8%, 0.6% or 0.5% (w/w) sodium polyacrylate. In one embodiment, the aqueous composition comprises between about 0.3% and 4%, between about 0.5% and 3%, between about 0.8% and 2.5%, or between about 0.5% and 1.5% (w/w) sodium polyacrylate.

In one embodiment, the rheology modifier comprises sodium polyacrylate and a co-emulsifier. As used herein the term “co-emulsifier” refers to a functional emulsifier present at lower concentrations than the primary functional ingredient responsible for/driving emulsification. For example, in the aqueous compositions described herein the co-emulsifier is present at a lower concentration (w/w) than sodium polyacrylate. Without being limited by theory, the co-emulsifier is believed to help stabilize the interface between the polyacrylate polymer, oil and water. In a classical emulsion, the emulsion structure if formed primarily due to the presence of emulsifiers. In contrast, a homogeneous mixture of oil and water is achieved in the embodiments described herein by the use of a water-swelling polymer that structures the water phase.

In one embodiment, the co-emulsifier is water soluble. In one embodiment, the co-emulsifier has a high hydrophobic-lipophilic balance (HLB). For example, in one embodiment the co-emulsifier has an HLB value greater than 10, greater than 12, greater than 14, or greater than 16. In one embodiment, the co-emulsifier has a HLB between about 15 and 19. In one embodiment, the co-emulsifier is a non-ionic surfactant, optionally a polysorbate surfactant such as polysorbate 20 (Tween™ 20) that has a HLB of about 16.7. In one embodiment, the co-emulsifier is polysorbate 40 (Tween™ 40) that has a HLB of about 15.6. In one embodiment, the co-emulsifier is a non-ionic emulsifier. In one embodiment, the co-emulsifier is a Ceteareth-based emulsifier or a PEGylated emulsifier.

In one embodiment, the aqueous composition comprises less than about 0.3%, 0.25%, 0.2% 0.15%, 0.1%, 0.05%, 0.025%, or 0.02% (w/w) of the co-emulsifier. In one embodiment, the aqueous composition comprises between about 0.005% and 0.3%, between about 0.005% and 0.2%, or between about 0.01% and 0.15% (w/w) of the co-emulsifier. In one embodiment, the aqueous composition comprises between about 0.02% and 0.1% or between about 0.04% and 0.08% (w/w) of the co-emulsifier.

Optionally, the rheology modifier may include sodium polyacrylate, a co-emulsifier and one or more additional components selected from a carrier oil, such as ethylhexyl cocoate, and an emollient such as PPG-3 benzyl ether myristate. In one embodiment, the rheology modifier is ViscOptima SE available from Croda Inc. ViscOptima™ SE is described as containing 50-100% sodium polyacrylate, 25-50% ethylhexyl cocoate, 5-10% PPG-3 benzyl ether myristate and 1-5% Polysorbate 20. In one embodiment, rheology modifier comprises between 50-69% sodium polyacrylate, 25-50% ethylhexyl cocoate, 5-10% PPG-3 benzyl ether myristate and 1-5% Polysorbate 20.

In one embodiment, the aqueous composition comprises less than 5%, 4%, 3.5% or 3% (w/w) of the rheology modifier, optionally between 1% and 5% (w/w) of the rheology modifier or between 2% and 4% (w/w) of the rheology modifier. In one embodiment, the aqueous composition comprises between about 2.5% and 3.5% (w/w) of the rheology modifier, optionally ViscOptima™ SE.

In one embodiment, the aqueous composition comprises PPG-3 benzyl ether ethylhexanoate, available commercially as Crodamol™ SFX. In one embodiment, the aqueous composition comprises less than 5%, 4%, 3.5%, or 3% (w/w) PPG-3 benzyl ether ethylhexanoate. In one embodiment, the aqueous composition comprises between 1% and 5% (w/w), between 2% and 5% (w/w) or between 2% and 4% (w/w) PPG-3 benzyl ether ethylhexanoate. In one embodiment, the aqueous composition comprises between about 2.5% and 3.5% (w/w) PPG-3 benzyl ether ethylhexanoate, optionally between about 2.8% and 3.3% (w/w) PPG-3 benzyl ether ethylhexanoate.

In one embodiment, the aqueous composition comprises glycerin. In one embodiment, the aqueous composition comprises less than 5% (w/w) glycerin. In one embodiment, the aqueous composition comprises between 1% and 5% (w/w) glycerin, between 2% and 5% (w/w) glycerin or between 2% and 4% (w/w) glycerin. In one embodiment, the aqueous composition comprises between about 2.5% and 3.5% (w/w) glycerin, optionally between about 2.8 and 3.3% (w/w) glycerin.

In one embodiment, the formulation comprises one or more anti-microbial agents. In one embodiment, the anti-microbial agent comprises one or more parabens, potassium sorbate, or phenoxyethanol and ethylhexylglycerin (Euxyl™ 9010). The use of potassium sorbate as an anti-microbial agent was determined to be particularly effective for reducing the growth or microbes such as bacteria, yeast and or fungi within the products described herein. In one embodiment, the aqueous composition comprises less than 1%, 0.5%, 0.2% or 0.1% (w/w) of the anti-microbial agent. In one embodiment, the aqueous composition comprises between 0.01% and 1% (w/w) of the anti-microbial agent, optionally between about 0.02% and 0.2% or between about 0.05% and 0.15% (w/w) of the anti-microbial agent. Preferably, the anti-microbial agent is present in the aqueous composition at a concentration sufficient to prohibit microbial growth according to USP<51> Antimicrobial Effectiveness Test, and/or USP <61> or <USP <62> Microbial Examination of Non-Sterile Products test.

The compositions described here are aqueous compositions and contain a significant amount of water relative to other components. In one embodiment, the aqueous composition comprises more than 50%, 60%, 70%, 80%, 85% or 90% (w/w) water. In one embodiment, the aqueous compositions described herein contain between 80% and 95% or between 85% and 95% (w/w) water.

As set out in the Examples, the characteristics of the aqueous compositions and associated oil-containing formulations comprising a rheology modifier as described herein are improved by the presence of glycerin and PPG-3 benzyl ether ethylhexanoate.

In one embodiment, the composition comprises, consists essentially of, or consists of: PPG-3 benzyl ether ethylhexanoate, a rheology modifier, glycerin, water and optionally one or more antimicrobial agents, coloring agents or fragrance agents.

In one embodiment, the aqueous composition comprises, consists essentially of, or consists of:

between 1% and 5% (w/w) PPG-3 benzyl ether ethylhexanoate;

between 1% and 5% (w/w) of the rheology modifier;

between 1% and 5% (w/w) glycerin; and optionally between 0.01% and 1% (w/w) of an anti-microbial agent.

In one embodiment, the rheology modifier comprises sodium polyacrylate and a co-emulsifier such as polysorbate 20. In one embodiment, the rheology modifier further comprises a carrier oil and an emollient. In one embodiment, the rheology modifier comprises an emollient that is not PPG-3 benzyl ether ethylhexanoate.

In a preferred embodiment, the aqueous composition comprises, consists essentially of, or consists of:

between 2% and 4% w/w PPG-3 benzyl ether ethylhexanoate;

between 2% and 4% w/w ViscOptima™ SE;

between 2% and 4% w/w glycerin;

between 0.01% and 1% w/w potassium sorbate; and

between 87% and 93.99% w/w water.

The aqueous compositions described herein are particularly suitably for the preparation of oil-containing cream formulations for topical use. As used herein, “oil” refers to a substance that is a viscous liquid at ambient temperatures and insoluble in water. Examples of oils include, but are not limited to, mineral oils, plant extracts such as essential oils, vegetable oils, and combinations thereof. Examples of vegetable oil include canola oil, corn oil, soybean oil, peanut oil, sunflower oil, safflower oil, avocado oil, grapeseed oil, and cottonseed oil. Essential oils include substances containing volatile aromatic compounds prepared from plant material, using techniques such as distillation, cold pressing, or solvent extraction to produce a viscous liquid that is insoluble in water. In one embodiment, the essential oil is prepared from cannabis, tea tree (melaleuca), frankincense, lavender, Melissa, sandalwood, cassia, cinnamon bark, clove, oregano, thyme, bergamot, black pepper, eucalyptus, ginger, and/or peppermint.

Additional examples of plant extracts suitable for use with the embodiments described herein include extracts made from plants with medicinal properties such as cannabis. In one embodiment, the plant extract comprises one or more cannabinoids and/or terpenes. In one embodiment, the plant extract is cannabidiol (CBD) oil. Cannabis plant extracts containing CBD may be prepared using methods known in the art such as solvent extraction or supercritical CO2 extraction to produce CBD containing oils suitable for use with the products described herein.

Optionally, in one embodiment the oil may comprise an oil solvent to which one or more non-polar substances are added. For example, the oil may include one or more non-polar medicinal ingredients dissolved in oil. In some embodiments, the oil may comprise a blend of oils and/or plant extracts such as a blend of a vegetable oil and a plant extract.

In one embodiment, the oil is a cannabis oil (cannabis plant extract) diluted with a carrier oil. In one embodiment, the cannabis oil is a CBD oil that contains less than about 7.5, 7, 6, 5, 4, 3, 2.5, 2, or 1 mg/ml CBD. In one embodiment, the oil is a CBD oil comprising between about 1 mg/ml and 5 mg/ml CBD. Optionally, the cannabis oil may contain ethanol as a residue from solvent extraction or other impurities like waxes and chlorophylls. In one embodiment, the cannabis oil is a CBD oil that contains less than 10 ppm THC. In one embodiment the oil contains synthetic CBD diluted in a carrier oil and/or CBD obtained from hemp.

As used herein “cream formulation” refers to colloidal suspension of an oil and an aqueous composition for topical application to the skin or a mucous membrane. In one embodiment, the colloidal suspension is an emulsion such as a gel emulsion. In one embodiment, the cream formulation has a higher relative amount of water relative to oil, i.e. it can be characterized as an oil-in-water cream formulation. In one embodiment, the cream formulations described herein have a viscosity of less than about 70,000, 65,000, 60,000, or 50,000 cps at 25° C. In one embodiment, the cream formulations have a viscosity of between about 40,000 and 70,000 cps at 25° C., or between about 40,000 and 65,000 cps at 25° C.

In a preferred embodiment, the cream formulation comprises, consists essentially of, or consists of an aqueous composition as described herein and an oil. In one embodiment, the oil comprises an essential oil or plant extract. In one embodiment, the oil comprises one or more medicinal ingredients, optionally within an essential or plant extract. As used herein, “medicinal ingredient” refers to a substance or mixture of substances used in the treatment, cure, prevention or suppression of a medical condition such as but not limited to a disease or disorder. A medicinal ingredient may also be used in the treatment, cure prevention or suppression of any symptoms associated with medical condition. In one embodiment, the medicinal ingredient is a cannabinoid and/or terpene.

As used herein, the term “effective amount” or “therapeutically effective amount” means an amount effective, at dosages and for periods of time necessary to achieve the desired result. For example, in the context of treating a medical condition such as pain, an effective amount is an amount that, for example, reduces the sensation of pain by the subject. Effective amounts may vary according to factors such as the disease state, age, sex and/or weight of the subject. The amount of a given medicinal ingredient that will correspond to such an amount will vary depending upon various factors, such as the given medicinal ingredient, the type of medical condition, the identity of the subject being treated, and the like, but can nevertheless be routinely determined by one skilled in the art.

As demonstrated in the Examples, the aqueous composition described have a high oil-load capacity. In one embodiment, the cream formulation comprises less than about 30%, 25%, 20% or 18% (w/w) oil. In one embodiment, the cream formulation comprises between about 0.5% and 30% (w/w) oil, between about 1% and 20% oil, or between about 5% and 18% oil. In one embodiment, the cream formulation comprise about 1%, 2%, 3%, 4%, 5%, 6%, 7%, 8%, 9%, 10%, 11%, 12%, 13%, 14%, 15%, 16%, 17%, 18%, 19% or 20% (w/w) oil. In one embodiment, the cream formulation comprises between about 10% and 18% or between about 12% and 18% (w/w) oil. In one embodiment, the cream formulation comprises between about 82% and 99% (w/w) of the aqueous composition and between about 1% and 18% (w/w) of the oil. In one embodiment, the cream formulation comprises between about 85% and 99% (w/w) of the aqueous composition and between about 1% and 15% (w/w) of the oil.

Also provided are methods for preparing a formulation comprising an aqueous composition and an oil as described herein. In one embodiment, the method comprises providing an aqueous composition and combining the aqueous composition and an oil. Various methods known in the art for physically combining two or more substances may be used to form the cream formulations, such as mixing and/or mechanically agitating. This may include manually or mechanically mixing the aqueous composition with the oil, optionally by using a stirring implement such as a stirring rod, beater, whisk, spoon, spatula or a similar object.

As demonstrated in the Examples, the aqueous compositions described herein advantageously have a high oil-load capacity. Furthermore, the formulations can be prepared without the need for heating and/or extended mixing. In one embodiment, combining the aqueous composition and the oil comprises combining between about 82% and 99% (w/w) of the aqueous composition and between about 1% and 18% (w/w) of the oil, between about 80% and 99% w/w of the aqueous composition and between about 1% and 18% w/w of the oil, or between about 75% and 99.9% of the aqueous composition and between about 0.1% and 25% (w/w) of the oil.

In one embodiment, the method comprises combining the aqueous composition and the oil without heating. For example, in one embodiment the method comprises combining the aqueous composition and the oil at a temperature of less than less than about 30° C., less than about 25° C., or at room temperature or ambient temperature. In one embodiment, the method comprises mixing the aqueous composition and the oil at a temperature between 15° C. and 25° C.

The formulations described herein can be readily prepared by mixing the aqueous composition with one or more oils. One advantage of the formulations is that they can be prepared outside of a production facility without extended mixing or high shear mixing. In one embodiment, the method comprises mixing the aqueous composition and the oil under low shear conditions.

In one embodiment, the formulations are prepared by manually mixing the aqueous composition and the oil to obtain a formulation that appears homogenous. For example, in one embodiment, the method comprises manually mixing the aqueous composition and the oil for less than 2 minutes, less than 90 seconds, less than 1 minute, or less than 30 seconds to obtain a formulation that appears homogenous. In one embodiment, manually mixing comprises stirring the aqueous composition and the oil with e.g. a stirring road or spatula for less for less than about 45, 30, 25 or 20 revolutions per minute.

Optionally, a consumer or an end-user performs the step of combining the aqueous composition and the oil to prepare the cream formulation prior to use. Advantageously, this allows the consumer or end-user to tailor specific formulations with the addition different oils or concentrations of oils.

In one embodiment, the products described herein are useful for the treatment of a medical condition in a subject in need thereof, such as by the topical application of a cream formulation containing one or more medicinal ingredients.

For example, cream formulations containing CBD oil may be used for the treatment of a variety of medical conditions, including but not limited to, pain, inflammation, pruritus, acne, tendonitis, eczema, psoriasis, cramps, rheumatism, arthritis, PTSD, IBS, sleep disorders, multiple sclerosis, headache and/or migraine. In one embodiment, cream formulations containing CBD as described herein may be used to treat one or more medical conditions identified in Table 8. As set out in the Examples, survey data from subjects who used the aqueous formulation to prepare CBD-oil containing cream formulations reported that the cream was “very effective” or “somewhat effective” for the treatment of a number of different medical conditions. Alternately or in addition, the cream formulation may contain one or more medicinal ingredients other than CBD and be useful for the treatment of medical conditions associated with therapeutic efficacy for the medicinal ingredient.

Accordingly, in one embodiment there is provided a method of treating a subject with a medical condition comprising administering to the subject a therapeutically effective amount of a cream formulation as described herein. Also provided is the use of a cream formulation as described herein for the treatment of a subject with a medical condition. In one embodiment, the cream formulation is for topical administration or use such as by applying and rubbing a therapeutically effective amount of the cream formulation onto the surface of the skin.

In one embodiment, the cream formulation contains a therapeutically effective amount of a medicinal ingredient, optionally in the form of an oil added to the aqueous composition. For example, in one embodiment the cream formulation contains CBD, optionally from about 0.01 to 1 mg/ml CBD, or from about 0.05 to 0.5 mg/ml CBD.

In one embodiment, the method comprises preparing the cream formulation prior to its use or administration using a method as described herein. For example, in one embodiment a consumer and/or end-user (such as a subject with a medical condition) performs the steps of preparing the cream formulation by, for example, mixing the aqueous composition and an oil. In one embodiment, the cream formulation may be self-administered or used by the consumer and/or end-user. For example, in one embodiment a subject with a medical condition performs the steps of preparing the cream formulation prior to self-administration or use of the formulation by the subject with the medical condition.

In another embodiment, there is provided a kit suitable for preparing a cream formulation as described herein. In one embodiment, the kit comprises an aqueous composition and one or more oils. In one embodiment, the kit further comprises instructions for combining the aqueous composition and the oil. In one embodiment, the aqueous composition and the oil are in separate containers. In one embodiment, the kit comprises a stirring implement such as a stirring rod or similar object for mixing the aqueous composition and the oil. In one embodiment, the kit comprises a plurality of different oils, wherein each oil comprises a different medicinal ingredient or concentration of medicinal ingredients. In one embodiment, the kit comprises a CBD oil. Optionally, the CBD in the oil is obtained from cannabis or hemp or is synthetic CBD diluted in a carrier oil. In one embodiment, the CBD oil contains less than 10 ppm THC.

Also provided are methods for the treatment of a subject with a medical condition, comprising providing to the subject a kit as described herein for the preparation of a cream formulation, wherein the subject prepares and administers a cream formulation comprising a medicinal ingredient. In one embodiment, the medicinal ingredient is a natural or synthetic plant extract such as cannabis oil.

### EXAMPLES

### Classical Cream Emulsions

Formulations using classical emulsifying agents such as glycerol stearate, PEG-100 stearate, Tweens and/or Spans as the primary emulsifiers were prepared as shown in Tables 1-3 by heating the oil and water phase prior to mixing under shear and allowing 24 hours for cooling.

TABLE 1
Classical emulsion prepared with Cetearyl alcohol, Glycerol stearate and
PEG-100 stearate and ~10% oil.

| Material | Role | Quantity (g) | % |
|---|---|---|---|
| Jojoba oil | Oil phase | 12 | 10 |
| Water | Aqueous phase | 90 | 75 |
| Xanthan gum | Water binding agent | 0.18 | 0.15 |
| Glycerin | Emollient | 6 | 5 |
| Myristyl myristate | Emollient | 4.8 | 4 |
| Cetearyl alcohol | Emulsifier; thickener | 1.2 | 1.0 |
| Glycerol stearate | Emulsifier | 4.37 | 3.64 |
| PEG-100 stearate | Emulsifier | 1.46 | 1.21 |

TABLE 2
Classical emulsion prepared with Span 80 and Tween 80 and ~10% oil.

| Material | Role | Quantity (g) | % |
|---|---|---|---|
| Jojoba oil | Oil phase | 11.4 | 9.5 |
| Jojoba flakes | Oil binding; thickener | 0.2 | 0.17 |
| Water | Aqueous phase | 90.4 | 75.33 |
| Glycerin | Emollient | 6 | 5 |
| Myristyl myristate | Emollient | 6 | 5 |
| Cetearyl alcohol | Emulsifier; thickener | 1.8 | 1.5 |
| Span 80 | Emulsifier | 2.86 | 2.4 |
| Tween 80 | Emulsifier | 1.34 | 1.1 |

TABLE 3
Classical emulsion prepared with Cetearyl alcohol, Glycerol stearate and
PEG-100 stearate and −3% oil.

| Material | Role | Quantity (g) | % |
|---|---|---|---|
| Jojoba oil | Oil phase | 3.5 | 2.9 |
| Water | Aqueous phase | 102.5 | 85 |
| Xanthan gum | Water binding agent | 0.15 | 0.13 |
| Glycerin | Emollient | 4 | 3.3 |
| Myristyl myristate | Emollient | 3.5 | 2.9 |
| Squalene | Emollient, moisturizer | 2 | 1.7 |
| Cetearyl alcohol | Emulsifier; thickener | 1.50 | 1.25 |
| Glycerol stearate | Emulsifier | 1.82 | 1.52 |
| PEG-100 stearate | Emulsifier | 1.03 | 0.86 |

On skin testing of the cream formulations set out in Tables 1 and 2 resulted in an oily feel at an oil concentration of ˜10% w/w. Cream formulations with ˜3% oil as set out in Table 3 did not have an oily skin feel on application however the relatively low-oil concentration is unsuitable for high-oil creams such as topical CBD creams.

### Identification of Aqueous Compositions for Oil-Containing Cream Formulations

A series of experiments were performed to identify aqueous compositions to which oil could be added later or reconstituted to prepare homogenous cream formulations suitable for topical use without heating and/or extended mixing. High oleic sunflower oil, which is commonly used as a carrier oil for CBD oil preparations, was used as an exemplary oil for testing purposes.

Experiments were conducted using a rheology modifier containing the water-swelling polymer sodium polyacrylate and a co-emulsifier. Without being limited by theory, it is believed that the water-swelling polymer swells upon contact with water, thereby reducing the amount of water available for interaction with the oil phase. The resulting hydrogels form fluid networks that permit the addition of oils to the aqueous composition without the need for heat or extended mixing.

ViscOptima™ SE is a rheology modifier available from Croda Inc. (Snaith, UK) containing sodium polyacrylate, ethylhexyl cocoate, PPG-3 Benzyl Ether Myristate and Polysorbate 20. Sodium polyacrylate is a superabsorbent polymer that has the ability to absorb as much as 100 to 1000 times its mass in water. ViscOptima™ SE is described by the manufacturer as containing: 50-100% sodium polyacrylate; 25-50% ethylhexyl cocoate; 5-10% PPG-3 Benzyl Ether Myristate; and 1-5% polysorbate 20.

ViscOptima™ SE was observed to form networks that were extremely fluid, and readily allowed for the incorporation of relatively high levels of oil into the existing composition by manually mixing without the addition of heat.

Additional testing was performed by adding 8 mL of oil to 52 mL of the aqueous composition containing various amounts of ViscOptima™ SE as shown in Table 4. The resulting cream formulations had an oil content of about 13.3% (v/v); accounting for the density of each ingredient about 7.32 g. of oil was added to 52.56 g. of the aqueous composition such that the formulations have an oil content of about 12.3% (w/w). As shown in Table 4, at least about 1% w/w (relative to water; prior to the addition of oil) ViscOptima™ SE was necessary to show any noticeable rheological effects (thickening) of the aqueous composition. Aqueous compositions containing levels of ViscOptima™ SE above 7.5% w/w (prior to the addition of oil) were observed to be “gunky” and exhibited some phase separation after the addition and mixing of oil. At about 5% ViscOptima™ SE (w/w), the composition had a very thick consistency and extended mixing was required to incorporate the oil in a homogenous cream. However, aqueous compositions having between 2% and 4% ViscOptima™ SE (prior to the addition of oil) were found to result in creams with a preferred consistency for topical use and exhibited rapid mixing with oil and formed homogenous cream formulations without oil separation.

TABLE 4
Effect of various concentrations of ViscOptima ™ SE on aqueous compositions
for preparing cream formulations containing high levels of oil (~12.3% (w/w)).

| ~0.5% (w/w) | ~1% (w/w) | ~2% (w/w) | ~3% (w/w) | ~4% (w/w) | ~5% (w/w) | ~7.5% (w/w) |
|---|---|---|---|---|---|---|
| Little or no | Low viscosity | Suitable | Suitable | Suitable | Thick | Very thick |
| thickening; | consistency; | consistency; | consistency; | consistency; | consistency; | consistency; |
| poor mixing | extended mixing | good mixing | good mixing | good mixing | extended mixing | phase separation |
| with oil | required to | with oil | with oil | with oil | required to | observed; |
| | incorporate oil | | | | incorporate oil | |

While ViscOptima™ SE contains an emollient (PPG-3 Benzyl Ether Myristate) and has been described as producing a desirable skin feel, testing showed that cream formulations containing ViscOptima™ SE and relatively high levels of oil (>12%) did not result in a desirable skin feel and/or otherwise have properties suitable for topical use. Additional experiments were therefore performed in order to try and identify compositions containing ViscOptima™ SE suitable for topical use with high levels of oil, such as for preparing CBD cream formulations.

### Qualitative Screening and Sensory Development of Topical Formulations

Key sensory requirements for topical formulations are that they do no not feel oily, and that they are absorbed rapidly into the skin. A greasy skin feel implies that the cream is not absorbing. Rather, it is sitting on the surface of the skin, which diminishes the therapeutic efficacy of the product. Furthermore, a non-greasy cream requires little spreading/rubbing. Low application times are ideal since it minimizes contact and carryover to the hand. Large carryover means that less of the oil or medicinal ingredient is available for absorption.

Aqueous compositions containing ViscOptima™ SE and high oleic sunflower oil were tested along with various components in order to try to improve the sensory characteristics and skin absorption of high-oil cream formulations.

Results from testing high-oil cream formulations comprising 3% ViscOptima™ as well a variety of different emollients and glycerin are shown in Table 5.

TABLE 5
Sensory evaluation of the use of different emollients for improving the
characteristics of cream formulations containing ViscOptima SE ™ and high levels of
oil (12.3% w/w).

| ViscOptima SE | Glycerin | Emollient | Evaluation |
|---|---|---|---|
| ~3% | 0% | — | Oily skin-feel |
| ~3% | ~3% | Isopropyl myristate | Dry skin-feel after product application, |
| | | | excessive spreading required for |
| | | | application |
| ~3% | ~3% | cetyl alcohol | Greasy skin-feel upon application |
| ~3% | ~3% | PPG-3 Benzyl Ether | Dry skin-feel after product application, |
| | | Ethylhexanoate | minimal spreading required for |
| | | | application |
| ~3% | ~3% | C12-15 alkyl benzoate | Oily residue after application |
| ~3% | ~3% | squalene | Excessively greasy skin-feel upon |
| | | | application |
| ~3% | ~3% | myristyl myristate | Dry and heavy skin feel; complicates |
| | | | manufacturing process since requires |
| | | | melting |

In particular, the use of the emollient Crodamol™ SFX (PPG-3 Benzyl Ether Ethylhexanoate) in combination with glycerin resulted in improved skin feel and absorption characteristics relative to the other emollients or the use of ViscOptima™ SE alone. Remarkably, these improvements were obtained without significantly impairing the ability of the formed composition to incorporate or reconstitute high levels of oil without heating and/or extended mixing.

Additional on-skin testing demonstrated that the cream formulations became oily and exhibited less advantageous characteristics for topical use when the levels of PPG-3 Benzyl Ether Ethylhexanoate (Crodamol™ SFX) in the aqueous composition exceeded 5% (w/w). On-skin testing also demonstrated that the formulations became sticky and exhibited less advantageous characteristics for topical use when the levels of the glycerin in the aqueous composition exceeded 5% (w/w). Beneficial improvements in the sensory characteristics of the cream formulations containing high levels of oil (˜12%) were also observed with the addition of at least 2% Crodamol™ SFX and at least 2% glycerin.

### Stability Testing

Aqueous compositions that passed sensory evaluation were subject to freeze-thaw cycle stability testing. This involved freezing samples containing ˜3% ViscOptima™ SE, ˜3%% glycerin and ˜3% PPG-3 Benzyl Ether Ethylhexanoate overnight, following by microwaving until molten the following day. Samples were tested both with and without the addition of oil (12.3% w/w). This was repeated three (3) times. Between each cycle, phase separation was monitored. If phase separation was observed, the product was re-mixed to determine if it would reform after separation. Samples containing ˜3% ViscOptima™ SE, ˜3% glycerin and ˜3% PPG-3 Benzyl Ether Ethylhexanoate were observed to be stable both with and without the addition of oil. Samples with or without oil that were frozen and subsequently molten were capable of readily incorporating oil by manual mixing at room temperature.

### Oil-Load Capacity

Additional testing of compositions comprising ViscOptima™ SE along with Crodamol™ SFX and glycerin were performed with a range of oil concentrations to determine the oil-load capacity. Compositions were prepared using a one-pot cold-mixing process as set out below and Table 6:

1. Weigh out potassium sorbate in mixing container
2. Add water to container
3. Add glycerin to container
4. Add Crodamol SFX to container
5. Add ViscOptima SE to container and mix until product is smooth and appears homogeneous.

TABLE 6
Ingredients and relative proportions of a preferred aqueous composition.

| Ingredient | | | Weight | Volume | % | % |
|---|---|---|---|---|---|---|
| Tradename | INCI | Description | (g) | (mL) | w/w | v/v |
| Water | Water | Aqueous phase | 47.79 | 47.79 | 90.93 | 91.91 |
| Glycerin | Glycerin | Humectant | 1.61 | 1.31 | 3.06 | 2.52 |
| Potassium | Potassium Sorbate | Anti-microbial | 0.05 | 0.04 | 0.09 | 0.07 |
| Sorbate | | | | | | |
| Crodamol ™ | PPG-3 Benzyl Ether | Emollient | 1.61 | 1.61 | 3.06 | 3.10 |
| SFX | Ethylhexanoate | | | | | |
| ViscOptima ™ | Sodium Polyacrylate, | Rheology | 1.5 | 1.25 | 2.85 | 2.40 |
| SE | Ethylhexyl Cocoate, | modifier/water | | | | |
| | PPG-3 Benzyl Ether | swelling | | | | |
| | Myristate and | polymer | | | | |
| | Polysorbate 20 | | | | | |

| | | | | |
|---|---|---|---|---|
| TOTAL | 52.56 | 52.00 | 100% | 100% |

Compositions were prepared and left to sit for at least 24 hours before mixing with various amounts of high oleic sunflower oil. Pre-determined amounts of oil were added to the compositions using a dropper at room temperature and mixed manually using a plastic spatula until the resulting formulations appeared homogenous.

Formulations having oil concentrations up to 18% (w/w) were easily mixed in less than about 1 minute with manual mixing (˜about 60-120 stirs per minute) using the spatula without the addition of heat. It was observed through on skin testing that cream formulations having up to 18% oil maintained a desirable skin feel and were topically absorbed.

Formulations having oil concentrations between 18-23% w/w typically required mixing for greater than about 1 minute (˜about 60-120 stirs per minute). These formulations also tended to feel greasy during on-skin testing.

Formulations having oil concentrations above 23% were observed to be too oily during on-skin application tests and required extended mixing (>2 minutes; (˜about 60-120 stirs per minute)) to achieve a homogenous appearance. Compatibility with Different Oils

Additional testing was performed to determine whether aqueous compositions comprising ViscOptima SE along with an emollient (Crodamol™ SFX) and a humectant (glycerin) exhibited desirable characteristics when mixed with oils other than high oleic sunflower oil. Aqueous compositions were prepared as set out in Table 6. Homogeneous cream formulations with ˜12% safflower oil were readily prepared by manual mixing without heating in less than 1 minute.

### Microbiological Testing

Microbiological testing was conducted according to USP <51> and USP (Antimicrobial Effectiveness Test) and USP <61> and <62> (Suitability Testing) in order to compare the effectiveness of 1) 0.09% potassium sorbate 2) 0.045% potassium sorbate and 0.045% Euxul™ 9010 and 3) 0.09% Euxul™ 9010 as an antimicrobial agent for prohibiting microbial growth in the aqueous composition described in Table 6.

Remarkably, potassium sorbate was determined to be as effective as effective as Euxul™ 9010 and/or the combination of Euxul™ 9010 and potassium sorbate. While all three samples passed the USP Antimicrobial Effectiveness Test and the USP Suitability Test, the Euxul™ 9010 sample had greater enumeration/growth between test periods than the other samples.

### Extended Stability Testing

Extended stability testing was performed on samples of the aqueous composition identified in Table 6. This involved holding the samples at 50° C. for 1 month. The viscosity and the pH of the cream were measured before and after incubation. As shown in Table 7, partial separation was observed, but the composition recombined upon mixing.

TABLE 7
Results of extended stability testing at 50° C.

| Test | Original Results | 1 month @ 50° C. |
|---|---|---|
| Appearance | Homogenous white cream | Slight separation |
| S.G. | 0.9981 | 1.0109 |
| pH | 5.78 | 5.65 |
| Viscosity | 67,000 cps | 14,000 cps |
| | (RVT 7/20/2000/ | (RVT 7/20/2000/7 @25° C.) |
| | 33.5 @25° C.) | |

Testing in Subjects with Different Medical Conditions

Subjects identified as users of CBD oil for medical purposes were provided the aqueous composition defined in Table 6 and instructions for preparing a cream formulation by mixing 52 ml of the aqueous composition with 8 ml of CBD oil to produce a cream with ˜12.3% (w/w) oil and apply as needed. A total of 479 respondents completed a survey asking them to evaluate the effectiveness of the cream formulation. As set out in Table 8, subjects indicated that the cream formulation was “somewhat effective” or “very effective” for a variety of conditions including arthritis (72% of respondents), PTSD (82% of respondents), fibromyalgia (80% of respondents) eczema (77% of respondents) and psoriasis (79% of respondents). 7/7 of respondents also indicated that the cream formulation was very effective for acne.

TABLE 8
Survey data from subjects using CBD-oil cream formulations prepared
using the aqueous composition defined in Table 6.

| | | % of Responses | % Responses |
|---|---|---|---|
| Condition | # Respondents | “Somewhat Effective” | “Very Effective” |
| Arthritis | 263 | 46% | 26% |
| Degenerative | 87 | 48% | 18% |
| Disc | | | |
| PTSD | 69 | 49% | 33% |
| Headaches | 62 | 41% | 30% |
| Tendonitis | 61 | 40% | 32% |
| Fibromyalgia | 49 | 51% | 29% |
| Migraines | 48 | 38% | 29% |
| Spinal Disc | 49 | 43% | 24% |
| Herniation | | | |
| Sleep | 38 | 45% | 32% |
| Disorder | | | |
| IBS | 22 | 45% | 23% |
| Eczema | 21 | 41% | 36% |
| Psoriasis | 18 | 61% | 18% |
| Acne | 7 | 0% | 100% |

1. An aqueous composition comprising:
a) a rheology modifier comprising sodium polyacrylate and a co-emulsifier;
b) PPG-3 benzyl ether ethylhexanoate;
c) glycerin; and
d) water.

2. The aqueous composition of claim 1, wherein the rheology modifier further comprises ethylhexyl cocoate and PPG-3 benzyl ether myristate.

3. The aqueous composition of claim 1, wherein the co-emulsifier is a non-ionic surfactant, optionally polysorbate 20.

4. The aqueous composition of claim 1, wherein the aqueous composition comprises between about 0.01% (w/w) and 0.25% or between about 0.02% and 0.25% (w/w) of the co-emulsifier.

5. The aqueous composition of claim 1, wherein the aqueous composition comprises between about 0.5% and 3.5% (w/w) or between 1% and 2.8% (w/w) sodium polyacrylate.

6. The aqueous composition of claim 1, wherein the aqueous composition further comprises between 0.01% and 1% (w/w) of an anti-microbial agent.

7. The aqueous composition of claim 6, wherein the anti-microbial agent is potassium sorbate.

8. The aqueous composition of claim 1, wherein the aqueous composition comprises between about 2% and 4% (w/w) PPG-3 benzyl ether ethylhexanoate.

9. The aqueous composition of claim 1, wherein the aqueous composition comprises between about 2% and 4% (w/w) glycerin.

10. The aqueous composition of claim 1, wherein the rheology modifier consists of:
between 50% and 100% (w/w) or between 50% and 69% (w/w) sodium polyacrylate;
between 25% and 50% (w/w) ethylhexyl cocoate;
between 5% and 10% (w/w) PPG-3 benzyl ether myristate; and
between 1% and 5% (w/w) polysorbate 20.

11. The aqueous composition of claim 10, wherein the aqueous composition comprises between 2% and 4% (w/w) of the rheology modifier.

12. The aqueous composition of claim 11, wherein the aqueous composition consists of the rheology modifier, PPG-3 benzyl ether ethylhexanoate, glycerin, water and an antimicrobial agent.

13. The aqueous composition of claim 1, wherein the aqueous composition comprises between 80% and 95% (w/w) water.

14. An aqueous composition consisting of:
between 2% and 4% (w/w) PPG-3 benzyl ether ethylhexanoate;
between 2% and 4% (w/w) ViscOptima™ SE;
between 2% and 4% (w/w) glycerin;
between 0.01% and 1% (w/w) potassium sorbate; and
between 87% and 93.99% (w/w) water.

15. A cream formulation comprising an oil and the aqueous composition of claim 1.

16. The cream formulation of claim 15, wherein the cream formulation comprises between 1% and 18% (w/w) oil, optionally between 8% and 18% (w/w) oil.

17. The cream formulation of claim 15, wherein the oil comprises cannabidiol (CBD).

18. A method of preparing a cream formulation, the method comprising:
providing the aqueous composition of claim 1; and
mixing the aqueous composition and an oil without heating to form the cream formulation.

19. A method of treating a subject with a medical condition, the method comprising topically administering to the subject a therapeutically effective amount of the cream formulation of claim 17.

20. A kit comprising:
an aqueous composition according to claim 1 and an oil,
wherein the aqueous composition and the oil are in separate containers.
